(19) United States
(12) Patent Application Publication
(10) Pub. No.: US 2023/0365937 A1
(43) Pub. Date: Nov. 16, 2023

(54) MESENCHYMAL STEM CELLS AND THEIR CULTURE

(71) Applicants: HADASIT MEDICAL RESEARCH SERVICES AND DEVELOPMENT LTD., Jerusalem (IL); NEUROGENESIS LTD., Givatayim (IL)

(72) Inventors: Ibrahim KASSIS, Haifa (IL); Dimitrios KARUSSIS, Jerusalem (IL); Tal GILAT, Givatayim (IL); Nir NETZER, Haifa (IL)

(21) Appl. No.: 18/033,629

(22) PCT Filed: Oct. 26, 2021
(86) PCT No.: PCT/IL2021/051267

§ 371 Date: Apr. 25, 2023

Related U.S. Application Data
(60) Provisional application No. 63/105,412, filed on Oct. 26, 2020.
(60) Provisional application No. 63/228,112, filed on Aug. 1, 2021.

(51) Int. Cl. C12N 5/0775; A61K 35/28; A61P 25/28

(52) U.S. Cl. CPC C12N 5/0663; A61K 35/28; A61P 25/28

(57) ABSTRACT

In vitro populations of enhanced mesenchymal stem cells (eMSCs) are provided. Pharmaceutical compositions comprising the eMSC populations as well as methods of culturing MSCs to produce the eMSC populations and use of the populations and compositions are also provided.

### CROSS-REFERENCE TO RELATED APPLICATIONS

This application claims the benefit of priority of U.S. Provisional Patent Application No. 63/105,412 filed Oct. 26, 2020, and U.S. Provisional Patent Application No. 63/228,112 filed Aug. 1, 2021 the contents of which are incorporated herein by reference in their entirety.

### FIELD OF INVENTION

The present invention is in the field of mesenchymal stem cells, and stem cell culture.

### BACKGROUND OF THE INVENTION

MSCs are multipotent progenitor cells capable of self-replication and are an important member of the bone marrow stem cell repertoire. These cells are described as nonhematopoietic stromal cells and their classical role is to support the process of hematopoiesis and HSC engraftment and to give rise to cells of mesodermal origin, such as osteoblasts, adipocytes and chondrocytes. MSCs are one of the known adult stem cells, and their use in various therapeutic modalities and for a variety of conditions/disease is currently a major area of investigation.

MSCs can be isolated from a variety of tissues including, bone marrow, adipose, dental pulp, placenta and umbilical cord by standard, well known protocols. The harvested MSCs can be directly administered to patients or can be cultured to increase their yield before they are administered. Further, MSCs do not elicit an immune response and therefore can be administered allogeneiclly to patients. To comply with the growing demand for mass scale production of MSC, techniques and culture media are continuously being developed for ex vivo expanding of these cells. A method of expanding harvested MSCs that also enhances the MSCs and makes them superior therapeutics is greatly needed.

### SUMMARY OF THE INVENTION

The present invention provides in vitro populations of enhanced mesenchymal stem cells (eMSCs), as well as pharmaceutical compositions comprising the eMSCs and methods of using same and of culturing MSCs to produce same.

According to a first aspect, there is provided an in vitro population of enhanced mesenchymal stem cells (eMSCs), comprising modulated expression of at least one protein selected from: PDGF, BDNF, beta-NGF, BMP-7, CNTF, EG-VEGF, FGF-4, FGF-7, GH, HB-EGF, HGF, IGFBP-1, Insulin, NGFR, NT-3, NT-4, PIGF, SCF, TGF-alpha, TGF-beta3, VEGFR2, VEGFR3, AR, BMP-4, GDF-15, GDNF, HGF, IGFBP-2, IGFBP-3, IGFBP-4, IGFBP-6, IGF-1, VEGF and VEGF-D.

According to another aspect, there is provided an in vitro population of eMSCs comprising a first subpopulation expressing at least one protein selected from: PDGF, BDNF, beta-NGF, BMP-7, CNTF, EG-VEGF, FGF-4, FGF-7, GH, HB-EGF, HGF, IGFBP-1, Insulin, NGFR, NT-3, NT-4, PIGF, SCF, TGF-alpha, TGF-beta3, VEGFR2, VEGFR3, AR, BMP-4, GDF-15, GDNF, HGF, IGFBP-2, IGFBP-3, IGFBP-4, IGFBP-6, IGF-1, VEGF and VEGF-D, wherein the first subpopulation comprise at least 10% of the in vitro population.

According to another aspect, there is provided an in vitro population of eMSCs comprising modulated surface expression of a protein selected from NTBA, SSEA-5, NPC (57D2), MUC-13, CD206, Notch1, Notch4, Notch3, NKp80, CD207, CD132, Jagged 2, GPR-56, CD66, DR3, CD85j, CD183, CD85h, CD319, GPR-19, CD24, HVEM, EGF-R, CD309, CD314, BTLA, and CD368.

According to another aspect, there is provided an in vitro population of eMSCs devoid of surface expression of at least one of protein selected from: CD271, SSEA-4, SSEA-3, CD133, CD106, CD146, CD54, CD58, CD62L and CD9.

According to another aspect, there is provided an in vitro population of eMSCs comprising at least a first subpopulation, wherein cells of the first subpopulation all express a surface protein selected from NTBA, SSEA-5, NPC (57D2), MUC-13, CD206, Notch1, Notch4, Notch3, NKp80, CD207, CD132, Jagged 2, GPR-56, CD66, DR3, CD85j, CD183, CD85h, CD319, GPR-19, CD24, HVEM, EGF-R, CD309, CD314, BTLA, and CD368, and wherein the first subpopulation comprises at least 30% of the eMSC population.

According to some embodiments, the population is characterized by enhanced pro-neurogenic capacity, enhanced immunosuppression, enhanced immunomodulation, enhanced anti-inflammatory capacity, enhanced pro-angiogenic capacity, enhanced neuroprotection, enhanced anti-apoptotic capacity, enhanced myelinogenic capacity, enhanced anti-fibrotic capacity, enhanced oligodendrocyte support, enhanced axonal support, enhanced neuronal differentiation or a combination thereof.

According to some embodiments, the modulated expression is enhanced expression and wherein the population comprises enhanced expression of at least 2 proteins.

According to some embodiments, the protein is selected from PDGF, BDNF, beta-NGF, BMP-7, CNTF, EG-VEGF, FGF-4, FGF-7, GH, HB-EGF, HGF, IGFBP-1, Insulin, NGFR, NT-3, NT-4, PIGF, SCF, TGF-alpha, TGF-beta3, VEGFR2, and VEGFR3.

According to some embodiments, the modulated expression is as compared to MSCs cultured in vitro under standard protocols.

According to some embodiments, the modulated expression is enhanced expression and comprises expression above a predetermined threshold.

According to some embodiments, the expression is protein secretion.

According to some embodiments, the in vitro population comprises at least 1×10\hat{ }7 MSCs.

According to some embodiments, the MSCs are human MSCs.

According to some embodiments, the MSCs are bone marrow derived MSCs.

According to some embodiments, the in vitro population comprises at least 90% MSCs.

According to some embodiments, the first subpopulation comprises:
a. at least 85% of the eMSCs and the surface protein is SSEA-5;
b. at least 80% of the eMSCs and the surface protein is NPC;
c. at least 75% of the eMSCs and the surface protein is MUC-13;
d. at least 70% of the eMSCs and the surface protein is CD206;
e. at least 70% of the eMSCs and the surface protein is Notch1;
f. at least 70% of the eMSCs and the surface protein is Notch 4;
g. at least 65% of the eMSCs and the surface protein is Notch 3;
h. at least 60% of the eMSCs and the surface protein is NTBA;
i. at least 55% of the eMSCs and the surface protein is NKp80;
j. at least 55% of the eMSCs and the surface protein is CD207;
k. at least 50% of the eMSCs and the surface protein is CD132;
l. at least 45% of the eMSCs and the surface protein is Jagged-2;
m. at least 45% of the eMSCs and the surface protein is GPR-56;
n. at least 45% of the eMSC and the surface protein is CD66;
o. at least 40% of the eMSCs and the surface protein is DR3;
p. at least 40% of the eMSCs and the surface protein is CD85j;
q. at least 40% of the eMSCs and the surface protein is CD183;
r. at least 35% of the eMSCs and the surface protein is CD85h;
s. at least 35% of the eMSCs and the surface protein is CD319;
t. at least 35% of the eMSCs and the surface protein is GPR-19;
u. at least 30% of the eMSCs and the surface protein is CD24;
v. at least 30% of the eMSCs and the surface protein is HVEM;
w. at least 30% of the eMSCs and the surface protein is EGFR;
x. at least 30% of the eMSCs and the surface protein is CD309;
y. at least 30% of the eMSCs and the surface protein is CD314;
z. at least 30% of the eMSCs and the surface protein is BTLA; or
aa. at least 30% of the eMSCs and the surface protein is CD368.

According to some embodiments, the population is devoid of surface expression of at least one of CD271, CD146 and SSEA-4.

According to some embodiments, the population is produced by a method of the invention.

According to another aspect, there is provided a pharmaceutical composition comprises an in vitro population of the invention.

According to some embodiments, the pharmaceutical composition is formulated for administration to a subject.

According to some embodiments, the pharmaceutical composition is formulated for intravenous or intrathecal administration.

According to another aspect, there is provided a method of culturing MSCs, the method comprising,
a. receiving a primary cell sample from a subject comprising MSCs;
b. isolating MSCs from the sample; and
c. culturing the MSCs in media for a time sufficient for increasing MSC number by at least 100%;
thereby culturing MSCs.

According to another aspect, there is provided a method of culturing MSCs, the method comprising,
a. receiving a primary cell sample from a subject comprising MSCs;
b. isolating MSCs from the sample; and
c. culturing the MSCs in media for a time sufficient for increasing MSC number by at least 100%;
wherein at least one of the following:
   i. the isolating comprises isolating mononuclear cells (MNCs) by Sepax separation;
   ii. the culturing comprises an initial seeding density of between 5000-8000 cells/square centimeter;
   iii. the media is NutriStem media supplemented with 5-15% human platelet lysate (HPL); or
   iv. a combination thereof;
      thereby culturing MSCs.

According to some embodiments, the primary cell sample is bone marrow aspirate.

According to some embodiments, the isolating comprises isolating mononuclear cells (MNCs).

According to some embodiments, the isolating MNC comprises performing a Ficoll density gradient, Sepax separation or both.

According to some embodiments, the method further comprises freezing the isolated MSCs and thawing the isolated MSCs.

According to some embodiments, the method further comprises washing the thawed

MSCs is a Dextran and albumin wash solution.

According to some embodiments, the wash solution comprises from 2-5% dextran 40 and 3-10% human Albumin.

According to some embodiments, the culturing comprises an initial seeding density of between 5000-8000 cells/square centimeter.

According to some embodiments, the media is NutriStem media supplemented with human platelet lysate (HPL).

According to some embodiments, the NutriStem media is supplemented with 7.5 to 15% HPL.

According to some embodiments, the HPL is about 10% HPL.

According to some embodiments, the media is further supplemented with non-essential vitamins, non-essential amino acids or both.

According to some embodiments, the non-essential vitamins are selected from Table 1.

According to some embodiments, the time is at least 4 days.

According to some embodiments, the culturing comprises removing 40-70% of the media and replacing it with an equal volume of fresh media about every 48 hours.

According to some embodiments, the method comprises removing about 50% of the media.

According to some embodiments, the method is for producing MSCs with modulated expression of at least one protein selected from: PDGF, BDNF, beta-NGF, BMP-7, CNTF, EG-VEGF, FGF-4, FGF-7, GH, HB-EGF, HGF, IGFBP-1, Insulin, NGFR, NT-3, NT-4, PIGF, SCF, TGF-alpha, TGF-beta3, VEGFR2, VEGFR3, AR, BMP-4, GDF-15, GDNF, HGF, IGFBP-2, IGFBP-3, IGFBP-4, IGFBP-6, IGF-1, VEGF and VEGF-D.

According to some embodiments, the method is for producing MSCs with modulated surface expression of a protein selected from NTBA, SSEA-5, NPC (57D2), MUC-13, CD206, Notch1, Notch4, Notch3, NKp80, CD207, CD132, Jagged 2, GPR-56, CD66, DR3, CD85j, CD183, CD85h, CD319, GPR-19, CD24, HVEM, EGF-R, CD309, CD314, BTLA, and CD368.

According to some embodiments, modulated in enhanced.

According to some embodiments, the method is for producing MSCs devoid of surface expression of at least one of protein selected from: CD271, SSEA-4, SSEA-3, CD133, CD106, CD146, CD54, CD58, CD62L and CD9.

According to some embodiments, the method is for producing MSCs comprising at least a first subpopulation, wherein cells of the first subpopulation all express a surface marker selected from NTBA, SSEA-5, NPC (57D2), MUC-13, CD206, Notch1, Notch4, Notch3, NKp80, CD207, CD132, Jagged 2, GPR-56, CD66, DR3, CD85j, CD183, CD85h, CD319, GPR-19, CD24, HVEM, EGF-R, CD309, CD314, BTLA, and CD368, and wherein the first subpopulation comprises at least 30% of the MSCs.

According to some embodiments, the method is for producing MSCs with pro-neurogenic capacity, enhanced pro-neurogenic capacity, enhanced immunosuppression, enhanced immunomodulation, enhanced anti-inflammatory capacity, enhanced pro-angiogenic capacity, enhanced neuroprotection, enhanced anti-apoptotic capacity, enhanced myelinogenic capacity, enhanced anti-fibrotic capacity, enhanced oligodendrocyte support, enhanced axonal support, enhanced neuronal differentiation or a combination thereof.

According to another aspect, there is provided an in vitro population of MSCs produced by a method of the invention.

According to another aspect, there is provided a method of treating a subject suffering from a condition treatable by MSC therapy, the method comprising administering to the subject the in vitro population of the invention or the pharmaceutical composition of the invention.

According to some embodiments, the condition is multiple sclerosis (MS).

According to some embodiments, the condition is amyotrophic lateral sclerosis (ALS).

According to some embodiments, the treating comprises decreasing neurofilament light chain (NfL) expression in the subject.

According to some embodiments, the decreasing is in serum of the subject.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A dot plot and bar graph of NfL levels in MS patients that received placebo (left) or eMSCs (right) by intrathecal injection. The bar graphs show the average concentration, and the dot plots the concentration for each individual. V3, V5 and V6 indicate patient visits and testing at 0-, 3- and 6-months post injection, respectively.

FIGS. 2A-2F: Bar graphs of secretion of (2A) BDNF, (2B) HGF, (2C) NT-3, (2D) CNTF, (2E) IGFBP-1 and (2F) PDGF from Lonza control MSC and eMSCs produced by culture with 5% HPL, 10% HPL and 10% HPL +vitamins.

### DETAILED DESCRIPTION OF THE INVENTION

The present invention, in some embodiments, provides populations of enhanced mesenchymal stem cells (eMSCs). The present invention further concerns a method of culturing MSCs to produce the eMSCs. Pharmaceutical compositions comprising the eMSCs are also provided.

### MSC Populations

By a first aspect, there is provided a population of mesenchymal stem cells (MSCs).

In some embodiments, the population is an in vitro population. In some embodiments, the population is an ex vivo population. In some embodiments, the population is a primary cell population. In some embodiments, the population is not a cell line population. In some embodiments, the population is derived from primary cells. In some embodiments, the population is not immortalized. In some embodiments, the population is a population of cultured primary MSCs. In some embodiments, the population is a mixed population. In some embodiments, the population is a homogenous population. In some embodiments, the population is a heterogeneous population.

In some embodiments, the population is an enhanced population (eMSCs). In some embodiments, the population is a non-naturally occurring population. In some embodiments, the population is an expanded population. In some embodiments, the population expresses at least one protein not expressed by naturally occurring MSCs. In some embodiments, the population expresses at least one protein at a higher level than is expressed by naturally occurring MSCs. In some embodiments, the protein is a surface protein. In some embodiments, the population comprises subpopulations of MSCs with defined expression profiles in a ratio not present in a naturally occurring MSC population.

In some embodiments, enhanced is as compared to naturally occurring MSCs. In some embodiments, enhanced is as compared to unmodified MSCs. In some embodiments, enhanced is as compared to MSCs cultured by a method known in the art. In some embodiments, the method known in the art is a standard culturing method. In some embodiments, the method known in the art is the method provided in Example 1. In some embodiments, enhanced is as compared to commercially available MSCs. In some embodiments, the commercially available MSCs are LONZA MSCs. In some embodiments, enhanced is as compared to MSC produced by a standard culturing method. In some embodiments, enhanced is as compared to MSCs cultured in vitro. In some embodiments, cultured in vitro is cultured under standard protocol. In some embodiments, the standard culturing method is the method disclosed hereinbelow in Example 1. In some embodiments, standard protocol is the protocol disclosed hereinbelow in Example 1.

In some embodiments, the MSC population is characterized by the enhancement. In some embodiments, enhanced comprises enhanced proliferation. In some embodiments, enhanced comprises a reduced doubling time. In some embodiments, enhanced comprises enhanced immunosuppression. In some embodiments, enhanced comprises enhanced immunomodulation. In some embodiments, enhanced comprises enhanced anti-inflammatory activity. In some embodiments, enhanced comprises enhanced anti-inflammatory capacity. In some embodiments, enhanced comprises enhanced anti-inflammatory potential. In some embodiments, enhanced anti-inflammatory comprises enhanced generation, transformation or conversion to an M2 phenotype. In some embodiments, enhanced anti-inflammatory comprises decreased generation, transformation or conversion to an M1 phenotype. In some embodiments, M1 and M2 refers to a phenotype of macrophages, astrocytes, microglia or a combination thereof. In some embodiments, M1 is pro-inflammatory. In some embodiments, M2 is pro-tolerogenic. In some embodiments, M2 is immunosuppressive. In some embodiments, enhanced comprises enhanced angiogenic potential. In some embodiments, the enhanced comprises enhanced pro-angiogenic capacity. In some embodiments, enhanced comprises enhanced neuroprotection. In some embodiments, enhanced neuroprotection comprises reduced axonal death. In some embodiments, enhanced neuroprotection comprises lowered levels of neurofilament light chain (NfL). In some embodiments, the lowered levels are in the cerebral spinal fluid (CSF). In some embodiments, enhanced comprises having neurogenic potential. In some embodiments, enhanced comprises having pro-neurogenic potential. In some embodiments, enhanced comprises having neurogenic capacity. In some embodiments, enhanced comprises having pro-neurogenic capacity. In some embodiments, enhanced comprises enhanced neurogenic potential. In some embodiments, enhanced comprises enhanced pro-neurogenic potential. In some embodiments, enhanced comprises enhanced neurogenic capacity. In some embodiments, enhanced comprises enhanced pro-neurogenic capacity. In some embodiments, enhanced comprises enhanced anti-apoptotic activity. In some embodiments, enhanced comprises enhanced anti-apoptotic capacity. In some embodiments, enhanced comprises enhanced potential. In some embodiments, enhanced comprises enhanced myelinogenic activity. In some embodiments, enhanced comprises enhanced myelinogenic capacity. In some embodiments, enhanced comprises enhanced myelinogenic potential. In some embodiments, enhanced comprises enhanced anti-fibrotic activity. In some embodiments, enhanced comprises enhanced anti-fibrotic capacity. In some embodiments, enhanced comprises enhanced anti-fibrotic potential. In some embodiments, enhanced comprises enhanced oligodendrocyte and/or axonal support. In some embodiments, support comprise nourishment and/or regeneration. In some embodiments, enhanced comprises enhanced differentiation to a neuronal phenotype.

In some embodiments, the MSCs are mammalian MSCs. In some embodiments, the MSCs are human MSCs. In some embodiments, the MSCs are bone marrow derived MSCs. In some embodiments, the MSCs are selected from bone marrow, adipose, dental pulp, placenta and umbilical cord derived MSCs. In some embodiments, the MSCs are derived from a healthy donor. In some embodiments, the MSCs are derived from a patient in need of MSC treatment. In some embodiments, the MSCs are derived from a patient suffering from a disease or condition that is treatable with MSCs. In some embodiments, the MSCs are autologous to a subject. In some embodiments, the MSCs are allogeneic to a subject. In some embodiments, the MSCs are heterologous to the subject.

In some embodiments, a disease treatable with MSCs is a disease or condition treatable by MSC therapy. In some embodiments, the disease treatable with MSCs is multiple sclerosis (MS). In some embodiments, the disease treatable with MSCs is amyotrophic lateral sclerosis (ALS) In some embodiments, treatable with MSCs is treatable with MSC therapy. In some embodiments, the disease treatable with MSCs graft versus host disease (GVHD). In some embodiments, the disease or condition is selected from a neurological disease, a muscular disease, an autoimmune disease, an inflammatory disease, a digestive disease, an energy homeostasis disease, a fibrotic disease, aging, radiation induced injury, cell transplant rejection and a proliferative disease.

In some embodiments, the disease or condition is a neurological disease. In some embodiments, the neurological disease is selected from brain cancer, cancer metastasis to the brain, multiple sclerosis (MS), amyotrophic lateral sclerosis (ALS), Alzheimer's disease, Parkinson's disease, neurological injury, radiation induced injury to the brain, hypoxic injury to the brain and Rett syndrome. In some embodiments, the neurological disease is MS. In some embodiments, the neurological disease is ALS. In some embodiments, the brain cancer is any one of an astrocytic tumor, a glioma, a medulloblastoma, a neuroblastoma and a meningioma. In some embodiments, the neurological disease is brain cancer. In some embodiments, the neurological disease is not brain cancer.

In some embodiments, the disease or condition is a muscular disease. In some embodiments, the muscular disease is selected from MS, ALS, a muscular dystrophy, muscle injury, muscle inflammation, cachexia and sarcopenia. In some embodiments, the muscular disease is MS. In some embodiments, the muscular dystrophy is Duchenne's muscular dystrophy (DMD), or Baker muscular dystrophy. In some embodiments, the muscular disease is ALS.

In some embodiments, the disease or condition is an autoimmune disease. In some embodiments, the autoimmune disease is selected from MS, diabetes, colitis, and Chron's disease. In some embodiments, the autoimmune disease is MS. In some embodiments, the autoimmune disease is ALS.

In some embodiments, the disease or condition is an energy homeostasis disease. In some embodiments, the energy homeostasis disease is diabetes. In some embodiments, the energy homeostasis disease is obesity.

In some embodiments, the disease or condition is a digestive disease. In some embodiments, the digestive disease is selected from irritable bowel syndrome (IBD), Chron's disease, and colitis.

In some embodiments, the disease or condition is aging. According to some embodiments, aging comprises at least one of skin aging, muscle aging, and brain aging.

In some embodiments, the disease or condition is a proliferative disease. In some embodiments, the proliferative disease is cancer. According to some embodiments, the cancer is any one of brain cancer, metastasis to the brain, lung cancer, breast cancer, colon cancer, pancreatic cancer, prostate cancer, and head and neck cancer. In some embodiments, the cancer is brain cancer.

In some embodiments, enhanced comprises modulated expression of at least one protein. In some embodiments, modulated expression is enhanced expression. In some embodiments, modulated expression is decreased expression. In some embodiments, expression is protein expression. In some embodiments, expression is mRNA expression. In some embodiments, expression is secretion. In some embodiments, the secretion of a protein from the MSC is modulated. In some embodiments, modulated expression comprises de-novo expression. In some embodiments, the MSCs express an enhancement protein. In some embodiments, the MSCs express at least one enhancement protein. In some embodiments, the protein is selected from the group consisting of BDNF, beta-NGF, BMP-7, b-FGF, EG-VEGF, FGF-4, FGF-7, GH, HB-EGF, IGFBP-1, Insulin, NGFR, NT-3, NT-4, PDGF, PIGF, SCF, TGF-alpha, TGF-beta3, VEGFR2, VEGFR3, AR, BMP-4, GDF-15, GDNF, HGF, IGFBP-2, IGFBP-3, IGFBP-4, IGFBP-6, IGF-1, VEGF, VEGF-D, BMP-5, and MCSFR. In some embodiments, the protein is selected from the group consisting of BDNF, beta-NGF, BMP-7, b-FGF, CNTF, EG-VEGF, FGF-4, FGF-7, GH, HB-EGF, HGF, IGFBP-1, Insulin, NGFR, NT-3, NT-4, PDGF, PIGF, SCF, TGF-alpha, TGF-beta3, VEGFR2, VEGFR3, AR, BMP-4, GDF-15, GDNF, HGF, IGFBP-2, IGFBP-3, IGFBP-4, IGFBP-6, IGF-1, VEGF, VEGF-D, BMP-5, and MCSFR. In some embodiments, a protein with decreased expression is selected from BMP-5 and MCSFR. In some embodiments, the protein is selected from the group consisting of BDNF, bFBF, beta-NGF, BMP-7, EG-VEGF, FGF-4, FGF-7, GH, HB-EGF, IGFBP-1, Insulin, NGFR, NT-3, NT-4, PDGF, PIGF, SCF, TGF-alpha, TGF-beta3, VEGFR2, VEGFR3, AR, BMP-4, GDF-15, GDNF, HGF, IGFBP-2, IGFBP-3, IGFBP-4, IGFBP-6, IGF-1, VEGF and VEGF-D. In some embodiments, the protein is selected from the group consisting of BDNF, bFGF, beta-NGF, BMP-7, CNTF, EG-VEGF, FGF-4, FGF-7, GH, HB-EGF, HGF, IGFBP-1, Insulin, NGFR, NT-3, NT-4, PDGF, PIGF, SCF, TGF-alpha, TGF-beta3, VEGFR2, VEGFR3, AR, BMP-4, GDF-15, GDNF, HGF, IGFBP-2, IGFBP-3, IGFBP-4, IGFBP-6, IGF-1, VEGF and VEGF-D. In some embodiments, the protein is selected from the group consisting of BDNF, bFGF, beta-NGF, BMP-7, EG-VEGF, FGF-4, FGF-7, GH, HB-EGF, IGFBP-1, Insulin, NGFR, NT-3, NT-4, PDGF, PIGF, SCF, TGF-alpha, TGF-beta3, VEGFR2, VEGFR3, BMP-4, GDF-15, HGF, IGFBP-2, IGFBP-3, IGFBP-4, IGFBP-6, IGF-1 and VEGF-D. In some embodiments, the protein is selected from the group consisting of BDNF, bFGF, beta-NGF, BMP-7, CNTF, EG-VEGF, FGF-4, FGF-7, GH, HB-EGF, HGF, IGFBP-1, Insulin, NGFR, NT-3, NT-4, PDGF-AA, PIGF, SCF, TGF-alpha, TGF-beta3, VEGFR2, VEGFR3, BMP-4, GDF-15, HGF, IGFBP-2, IGFBP-3, IGFBP-4, IGFBP-6, IGF-1 and VEGF-D. In some embodiments, the protein is selected from the group consisting of BDNF, BMP-7, EG-VEGF, FGF-4, FGF-7, GH, HB-EGF, IGFBP-1, Insulin, NGFR, NT-3, NT-4, PDGF, SCF, TGF-alpha, TGF-beta3, VEGFR2, VEGFR3, BMP-4, GDF-15, HGF, IGFBP-2, IGFBP-3, IGFBP-4, IGFBP-6, IGF-1 and VEGF-D. In some embodiments, the protein is selected from the group consisting of BDNF, BMP-7, CNTF, EG-VEGF, FGF-4, FGF-7, GH, HB-EGF, HGF, IGFBP-1, Insulin, NGFR, NT-3, NT-4, PDGF, SCF, TGF-alpha, TGF-beta3, VEGFR2, VEGFR3, BMP-4, GDF-15, HGF, IGFBP-2, IGFBP-3, IGFBP-4, IGFBP-6, IGF-1 and VEGF-D. In some embodiments, the protein is selected from the group consisting of BDNF, b-FGF, beta-NGF, BMP-7, EG-VEGF, FGF-4, FGF-7, GH, HB-EGF, IGFBP-1, Insulin, NGFR, NT-3, NT-4, PDGF, PIGF, SCF, TGF-alpha, TGF-beta3, VEGFR2, and VEGFR3. In some embodiments, the protein is selected from the group consisting of BDNF, b-FGF, beta-NGF, BMP-7, CNTF, EG-VEGF, FGF-4, FGF-7, GH, HB-EGF, HGF, IGFBP-1, Insulin, NGFR, NT-3, NT-4, PDGF, PIGF, SCF, TGF-alpha, TGF-beta3, VEGFR2, and VEGFR3. In some embodiments, the protein is selected from the group consisting of BDNF, BMP-7, EG-VEGF, FGF-4, FGF-7, GH, HB-EGF, IGFBP-1, Insulin, NGFR, NT-3, NT-4, PDGF, SCF, TGF-alpha, TGF-beta3, VEGFR2, and VEGFR3. In some embodiments, the protein is selected from the group consisting of BDNF, BMP-7, CNTF, EG-VEGF, FGF-4, FGF-7, GH, HB-EGF, HGF, IGFBP-1, Insulin, NGFR, NT-3, NT-4, PDGF, SCF, TGF-alpha, TGF-beta3, VEGFR2, and VEGFR3. In some embodiments, the protein is selected from the group consisting of BDNF, CNTF, HGF, IGFBP-1, NT-3, and PDGF. In some embodiments, the protein is selected from the group consisting of CNTF, IGFBP-1, NT-3, and PDGF. In some embodiments, the protein is selected from the group consisting of CNTF, IGFBP-1, and PDGF. In some embodiments, the protein is selected from the group consisting of IGFBP-1, and PDGF. In some embodiments, the MSCs overexpress the enhancement protein. In some embodiments, the MSCs comprise enhanced expression of at least one enhancement protein. In some embodiment, the MSCs comprise overexpression of at least one enhancement protein. In some embodiments, the MSCs comprise expression of at least one enhancement protein above a predetermined threshold. In some embodiments, the MSCs comprise de novo expression of at least one enhancement protein. In some embodiments, at least 1 is at least 2. In some embodiments, at least 1 is at least 3. In some embodiments, at least 1 is at least 5. In some embodiments, at least 1 is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35. Each possibility represents a separate embodiment of the invention.

In some embodiments, an enhanced protein is at least one enhanced protein. In some embodiments, an enhanced protein is a plurality of enhanced proteins. In some embodiments, the enhanced protein is brain-derived neurotrophic factor (BDNF). In some embodiments, the enhanced protein is nerve growth factor beta (beta-NGF, or bNGF). In some embodiments, the enhanced protein is basic fibroblast growth factor (bFGF or FGF2). In some embodiments, the enhanced protein is bone morphogenetic protein 7 (BMP-7). In some embodiments, the enhanced protein is ciliary neurotrophic factor (CNTF). In some embodiments, the enhanced protein is endocrine-gland-derived vascular endothelial growth factor (EG-VEGF). In some embodiments, EG-VEGF is prokineticin-1 (PROK1). In some embodiments, the enhanced protein is fibroblast growth factor 4 (FGF-4). In some embodiments, the enhanced protein is fibroblast growth factor 7 (FGF-7). In some embodiments, the enhanced protein is growth hormone (GH). In some embodiments, the enhanced protein is heparin-binding EGF-like growth factor (HB-EGF). In some embodiments, the enhanced protein is hepatocyte growth factor (HGF). In some embodiments, the enhanced protein is insulin like growth factor binding protein 1 (IGFBP-1). In some embodiments, the enhanced protein is insulin. In some embodiments, the enhanced protein is nerve growth factor receptor (NGFR). In some embodiments, the enhanced protein is neurotrophin 3 (NT-3). In some embodiments, the enhanced protein is neurotrophin 4 (NT-4). In some embodiments, the enhanced protein is stem cell factor (SCF). In some embodiments, the enhanced protein is platelet-derived growth factor (PDGF). In some embodiments, the enhanced protein is PDGF-AA. In some embodiments, PDGF is PDGF isoform PDGF-AA. In some embodiments, the enhanced protein is phosphatidylinositol glycan anchor biosynthesis class F protein (PIGF). In some embodiments, the enhanced protein is transforming growth factor alpha (TGFa). In some embodiments, the enhanced protein is TGF-beta3. In some embodiments, the enhanced protein is vascular endothelial growth factor receptor 2 (VEGFR2). In some embodiments, the enhanced protein is VEGFR3. In some embodiments, the enhanced protein is selected from Table 2. In some embodiments, the enhanced protein is selected from a protein provided in Table 2. In some embodiments, modulated is increased and the protein is selected from a protein provided in Table 2. In some embodiments, modulated is ectopically expressed and the protein is selected from a protein provided in Table 2. In some embodiments, the protein is a secreted protein and selected from a protein provided in Table 2.

In some embodiments, the enhanced protein is androgen receptor (AR). In some embodiments, the enhanced protein is bone morphogenetic protein 4 (BMP-4). In some embodiments, the enhanced protein is growth/differentiation factor-15 (GDF-15). In some embodiments, the enhanced protein is glial cell-derived neurotrophic factor (GDNF). In some embodiments, the enhanced protein is HGF. In some embodiments, the enhanced protein is IGFBP-2. In some embodiments, the enhanced protein is IGFBP-3. In some embodiments, the enhanced protein is IGFBP-4. In some embodiments, the enhanced protein is IGFBP-6. In some embodiments, the enhanced protein is insulin like growth factor 1 (IGF-1). In some embodiments, the enhanced protein is vascular endothelial growth factor (VEGF). In some embodiments, the enhanced protein is vascular endothelial growth factor D (VEGF-D). In some embodiments, the enhanced protein is selected from Table 3. In some embodiments, the enhanced protein is selected from a protein provided in Table 3. In some embodiments, modulated is increased and the protein is selected from a protein provided in Table 3. In some embodiments, the protein is a secreted protein and selected from a protein provided in Table 3.

In some embodiments, the enhanced protein is a surface protein. In some embodiments, the MSCs comprise modulated expression of a surface protein. In some embodiments, the MSCs comprise modulated surface expression of a surface protein. In some embodiments, the MSCs comprise expression of the surface protein. In some embodiments, the MSCs comprise a subpopulation that express the surface protein. In some embodiments, the subpopulation is a first subpopulation. In some embodiments, all cells of the subpopulation express a given surface protein. In some embodiments, the surface protein is a receptor. In some embodiments, the MSCs comprise at least 1, 2, 3, 4, 5, 6, 7, 8, 9, 10, 11, 12, 13, 14, 15, 16, 17, 18, 19, 20, 21, 22, 23, 24, 25, 26 or 27 subpopulations. Each possibility represents a separate embodiment of the invention.

In some embodiments, the surface protein is selected from Table 5. In some embodiments, the surface protein is selected from a protein provided in Table 5. In some embodiments, the surface protein is selected from SSEA-5, NPC (57D2), MUC-13, CD206, Notch1, Notch4, Notch3, NTBA, NKp80, CD207, CD132, Jagged 2, GPR-56, CD66, DR3, CD85j, CD183, CD85h, CD319, GPR-19, CD24, HVEM, EGF-R, CD309, CD314, BTLA, and CD368. In some embodiments, the surface protein is selected from NTBA, and NOTCH1. In some embodiments, the surface protein is selected from SSEA-5, NTBA, and NOTCH1. In some embodiments, the surface protein is selected from CD207, SSEA-5, NTBA, and NOTCH1. In some embodiments, the surface protein is selected from NPC, MUC13, CD207, SSEA-5, NTBA, and NOTCH1. In some embodiments, the surface protein is stage-specific embryonic antigen-5 (SSEA-5). In some embodiments, the surface protein is nuclear pore complex (NPC). In some embodiments, the surface protein is mucin 13 (MUC-13). In some embodiments, the surface protein is mannose receptor (CD206). In some embodiments, the surface protein is Notch1. In some embodiments, the surface protein is Notch4. In some embodiments, the surface protein is Notch3. In some embodiments, the surface protein is SLAM family member 6 (SLAMF6, CD352 or NTBA). In some embodiments, the surface protein is killer cell lectin-like subfamily F, member 1 (KLRF1 or NKp80). In some embodiments, the surface protein is c-type lectin domain family 4 member K (CD207). In some embodiments, the surface protein is interleukin-2 receptor subunit gamma (IL2RG or CD132). In some embodiments, the surface protein is Jagged 2. In some embodiments, the surface protein is G protein-coupled receptor 56 (TM7XN1 or GPR-56). In some embodiments, the surface protein is CD66. In some embodiments, the surface protein is death receptor 3 (DR3). In some embodiments, the surface protein is leukocyte immunoglobulin like receptor B1 (LILRB1, ILT2 or CD85j). In some embodiments, the surface protein is chemokine receptor CXCR3 (CXCR3 or CD183). In some embodiments, the surface protein is leukocyte immunoglobulin-like receptor subfamily A member 2 (LILRA2 or CD85h). In some embodiments, the surface protein is SLAM family member 7 (SLAMF7 or CD319). In some embodiments, the surface protein is GPR-19. In some embodiments, the surface protein is signal transducer CD24 (CD24). In some embodiments, the surface protein is HVEM. In some embodiments, the surface protein is epidermal growth factor receptor (EGF-R). In some embodiments, the surface protein is kinase insert domain receptor (KDR, VEGFR2, or CD309). In some embodiments, the surface protein is killer cell lectin like receptor K1 (KLRK1, NKG2D, or CD314). In some embodiments, the surface protein is B- and T-lymphocyte attenuator (BTLA). In some embodiments, the surface protein is C-type lectin 4D (CLEC4D or CD368).

In some embodiments, the MSC population comprises a first subpopulation that expresses at least one enhancement protein. In some embodiments, the MSC population comprises a first subpopulation that expresses at least one surface protein. In some embodiments, the MSC population comprises at least a first subpopulation that expresses at least one enhancement protein. In some embodiments, the MSC population comprises at least a first subpopulation that expresses at least one surface protein. In some embodiments, a subpopulation is characterized by expression of at least one enhancement protein. In some embodiments, a subpopulation is characterized by expression of at least one surface protein. In some embodiments, a subpopulation overexpresses, upregulates, or comprises enhanced expression of the at least one enhancement protein. In some embodiments, a subpopulation comprises at least 10, 20, 25, 30, 35, 40, 45, 50, 55, 60, 65, 70, 75, 80, 85, 90, 95, 97, 99 or 100% of the population. Each possibility represents a separate embodiment of the invention. In some embodiments, a subpopulation comprises at least 10% of the population. In some embodiments, a subpopulation comprises at least 15% of the population. In some embodiments, a subpopulation comprises at least 20% of the population. In some embodiments, a subpopulation comprises at least 25% of the population. In some embodiments, a subpopulation comprises at least 30% of the population. In some embodiments, a subpopulation comprises at least 35% of the population. In some embodiments, a subpopulation comprises at least 40% of the population. In some embodiments, a subpopulation comprises at least 45% of the population. In some embodiments, a subpopulation comprises at least 50% of the population. In some embodiments, a subpopulation comprises at least 55% of the population. In some embodiments, a subpopulation comprises at least 60% of the population. In some embodiments, a subpopulation comprises at least 65% of the population. In some embodiments, a subpopulation comprises at least 70% of the population. In some embodiments, a subpopulation comprises at least 75% of the population. In some embodiments, a subpopulation comprises at least 80% of the population. In some embodiments, a subpopulation comprises at least 85% of the population. In some embodiments, a subpopulation comprises at least 90% of the population. In some embodiments, a subpopulation comprises at least 95% of the population. In some embodiments, a subpopulation comprises less than 100% of the population. In some embodiments, a subpopulation comprises less than 100, 97, 95, 90, 85, 80, 75, 70, 60, 50, 40, 30, 25, or 20% of the population. Each possibility represents a separate embodiment of the invention. In some embodiments, the MSC population comprises a second subpopulation. In some embodiments, the second subpopulation expresses at least a second enhancement protein. In some embodiments, the second subpopulation expresses at least a second surface protein. In some embodiments, the second subpopulation is characterized by expression of at least one enhancement protein. In some embodiments, the second subpopulation is characterized by expression of at least one surface protein. In some embodiments, the first and second subpopulations are different subpopulations. In some embodiments, the first and second subpopulations are characterized by at least one different enhanced protein. In some embodiments, the first and second subpopulations are characterized by at least one different surface protein. In some embodiments, the subpopulations express a surface protein provided in Table 5 and make up at least the percent of the MSC population provided in Table 5 for that surface protein. In some embodiments, the subpopulations express NOTCH1 and NTBA. In some embodiments, the subpopulations express a surface protein selected from SSEA-5, NOTCH1 and NTBA. In some embodiments, the subpopulations express a surface protein selected from CD207, SSEA-5, NOTCH1 and NTBA. In some embodiments, the subpopulations express a surface protein selected from NPC, MUC13, CD207, SSEA-5, NOTCH1 and NTBA.

In some embodiments, the surface is SSEA-5 and the subpopulation is at least 85% of the MSCs. In some embodiments, the surface is SSEA-5 and the subpopulation is at least 80% of the MSCs. In some embodiments, the surface is SSEA-5 and the subpopulation is at least 75% of the MSCs. In some embodiments, the surface is SSEA-5 and the subpopulation is at least 70% of the MSCs.

In some embodiments, the surface is NPC and the subpopulation is at least 80% of the MSCs. In some embodiments, the surface is NPC and the subpopulation is at least 75% of the MSCs. In some embodiments, the surface is NPC and the subpopulation is at least 70% of the MSCs. In some embodiments, the surface is NPC and the subpopulation is at least 65% of the MSCs.

In some embodiments, the surface is MUC-13 and the subpopulation is at least 75% of the MSCs. In some embodiments, the surface is MUC-13 and the subpopulation is at least 70% of the MSCs. In some embodiments, the surface is MUC-13 and the subpopulation is at least 65% of the MSCs. In some embodiments, the surface is MUC-13 and the subpopulation is at least 60% of the MSCs.

In some embodiments, the surface is CD206 and the subpopulation is at least 70% of the MSCs. In some embodiments, the surface is CD206 and the subpopulation is at least 65% of the MSCs. In some embodiments, the surface is CD206 and the subpopulation is at least 60% of the MSCs. In some embodiments, the surface is CD206 and the subpopulation is at least 55% of the MSCs.

In some embodiments, the surface is Notch1 and the subpopulation is at least 70% of the MSCs. In some embodiments, the surface is Notch1 and the subpopulation is at least 65% of the MSCs. In some embodiments, the surface is Notch1 and the subpopulation is at least 60% of the MSCs. In some embodiments, the surface is Notch1 and the subpopulation is at least 55% of the MSCs.

In some embodiments, the surface is Notch4 and the subpopulation is at least 70% of the MSCs. In some embodiments, the surface is Notch4 and the subpopulation is at least 65% of the MSCs. In some embodiments, the surface is Notch4 and the subpopulation is at least 60% of the MSCs. In some embodiments, the surface is Notch4 and the subpopulation is at least 55% of the MSCs.

In some embodiments, the surface is Notch3 and the subpopulation is at least 65% of the MSCs. In some embodiments, the surface is Notch3 and the subpopulation is at least 60% of the MSCs. In some embodiments, the surface is Notch3 and the subpopulation is at least 55% of the MSCs. In some embodiments, the surface is Notch3 and the subpopulation is at least 50% of the MSCs.

In some embodiments, the surface is NTBA and the subpopulation is at least 60% of the MSCs. In some embodiments, the surface is NTBA and the subpopulation is at least 55% of the MSCs. In some embodiments, the surface is NTBA and the subpopulation is at least 50% of the MSCs. In some embodiments, the surface is NTBA and the subpopulation is at least 45% of the MSCs.

In some embodiments, the surface is NKP80 and the subpopulation is at least 55% of the MSCs. In some embodiments, the surface is NKP80 and the subpopulation is at least 50% of the MSCs. In some embodiments, the surface is NKP80 and the subpopulation is at least 45% of the MSCs. In some embodiments, the surface is NKP80 and the subpopulation is at least 40% of the MSCs.

In some embodiments, the surface is CD207 and the subpopulation is at least 55% of the MSCs. In some embodiments, the surface is CD207 and the subpopulation is at least 50% of the MSCs. In some embodiments, the surface is CD207 and the subpopulation is at least 45% of the MSCs. In some embodiments, the surface is CD207 and the subpopulation is at least 40% of the MSCs.

In some embodiments, the surface is CD132 and the subpopulation is at least 50% of the MSCs. In some embodiments, the surface is CD132 and the subpopulation is at least 45% of the MSCs. In some embodiments, the surface is CD132 and the subpopulation is at least 40% of the MSCs. In some embodiments, the surface is CD132 and the subpopulation is at least 35% of the MSCs.

In some embodiments, the surface is Jagged-2 and the subpopulation is at least 45% of the MSCs. In some embodiments, the surface is Jagged-2 and the subpopulation is at least 40% of the MSCs. In some embodiments, the surface is Jagged-2 and the subpopulation is at least 35% of the MSCs. In some embodiments, the surface is Jagged-2 and the subpopulation is at least 30% of the MSCs.

In some embodiments, the surface is GPR-56 and the subpopulation is at least 45% of the MSCs. In some embodiments, the surface is GPR-56 and the subpopulation is at least 40% of the MSCs. In some embodiments, the surface is GPR-56 and the subpopulation is at least 35% of the MSCs. In some embodiments, the surface is GPR-56 and the subpopulation is at least 30% of the MSCs.

In some embodiments, the surface is CD66 and the subpopulation is at least 45% of the MSCs. In some embodiments, the surface is CD66 and the subpopulation is at least 40% of the MSCs. In some embodiments, the surface is CD66 and the subpopulation is at least 35% of the MSCs. In some embodiments, the surface is CD66 and the subpopulation is at least 30% of the MSCs.

In some embodiments, the surface is DR3 and the subpopulation is at least 40% of the MSCs. In some embodiments, the surface is DR3 and the subpopulation is at least 35% of the MSCs. In some embodiments, the surface is DR3 and the subpopulation is at least 30% of the MSCs. In some embodiments, the surface is DR3 and the subpopulation is at least 25% of the MSCs.

In some embodiments, the surface is CD85j and the subpopulation is at least 40% of the MSCs. In some embodiments, the surface is CD85j and the subpopulation is at least 35% of the MSCs. In some embodiments, the surface is CD85j and the subpopulation is at least 30% of the MSCs. In some embodiments, the surface is CD85j and the subpopulation is at least 25% of the MSCs.

In some embodiments, the surface is CD183 and the subpopulation is at least 40% of the MSCs. In some embodiments, the surface is CD183 and the subpopulation is at least 35% of the MSCs. In some embodiments, the surface is CD183 and the subpopulation is at least 30% of the MSCs. In some embodiments, the surface is CD183 and the subpopulation is at least 25% of the MSCs.

In some embodiments, the surface is CD85h and the subpopulation is at least 35% of the MSCs. In some embodiments, the surface is CD85h and the subpopulation is at least 30% of the MSCs. In some embodiments, the surface is CD85h and the subpopulation is at least 25% of the MSCs. In some embodiments, the surface is CD85h and the subpopulation is at least 20% of the MSCs.

In some embodiments, the surface is CD319 and the subpopulation is at least 35% of the MSCs. In some embodiments, the surface is CD319 and the subpopulation is at least 30% of the MSCs. In some embodiments, the surface is CD319 and the subpopulation is at least 25% of the MSCs. In some embodiments, the surface is CD319 and the subpopulation is at least 20% of the MSCs.

In some embodiments, the surface is GPR-19 and the subpopulation is at least 35% of the MSCs. In some embodiments, the surface is GPR-19 and the subpopulation is at least 30% of the MSCs. In some embodiments, the surface is GPR-19 and the subpopulation is at least 25% of the MSCs. In some embodiments, the surface is GPR-19 and the subpopulation is at least 20% of the MSCs.

In some embodiments, the surface is CD24 and the subpopulation is at least 30% of the MSCs. In some embodiments, the surface is CD24 and the subpopulation is at least 25% of the MSCs. In some embodiments, the surface is CD24 and the subpopulation is at least 20% of the MSCs. In some embodiments, the surface is CD24 and the subpopulation is at least 15% of the MSCs.

In some embodiments, the surface is HVEM and the subpopulation is at least 30% of the MSCs. In some embodiments, the surface is HVEM and the subpopulation is at least 25% of the MSCs. In some embodiments, the surface is HVEM and the subpopulation is at least 20% of the MSCs. In some embodiments, the surface is HVEM and the subpopulation is at least 15% of the MSCs.

In some embodiments, the surface is EGFR and the subpopulation is at least 30% of the MSCs. In some embodiments, the surface is EGFR and the subpopulation is at least 25% of the MSCs. In some embodiments, the surface is EGFR and the subpopulation is at least 20% of the MSCs. In some embodiments, the surface is EGFR and the subpopulation is at least 15% of the MSCs.

In some embodiments, the surface is CD309 and the subpopulation is at least 30% of the MSCs. In some embodiments, the surface is CD309 and the subpopulation is at least 25% of the MSCs. In some embodiments, the surface is CD309 and the subpopulation is at least 20% of the MSCs. In some embodiments, the surface is CD309 and the subpopulation is at least 15% of the MSCs.

In some embodiments, the surface is CD314 and the subpopulation is at least 30% of the MSCs. In some embodiments, the surface is CD314 and the subpopulation is at least 25% of the MSCs. In some embodiments, the surface is CD314 and the subpopulation is at least 20% of the MSCs. In some embodiments, the surface is CD314 and the subpopulation is at least 15% of the MSCs.

In some embodiments, the surface is BTLA and the subpopulation is at least 30% of the MSCs. In some embodiments, the surface is BTLA and the subpopulation is at least 25% of the MSCs. In some embodiments, the surface is BTLA and the subpopulation is at least 20% of the MSCs. In some embodiments, the surface is BTLA and the subpopulation is at least 15% of the MSCs.

In some embodiments, the surface is CD368 and the subpopulation is at least 30% of the MSCs. In some embodiments, the surface is CD368 and the subpopulation is at least 25% of the MSCs. In some embodiments, the surface is CD368 and the subpopulation is at least 20% of the MSCs. In some embodiments, the surface is CD368 and the subpopulation is at least 15% of the MSCs.

In some embodiments, the MSCs are devoid of surface expression of at least one of protein selected from: SSEA-4, SSEA-3, CD133, CD106, CD146, CD271, CD54, CD58, CD62L and CD9. In some embodiments, the MSCs are devoid of surface expression of at least one of CD146, CD271 and SSEA-4. In some embodiments, the MSCs are devoid of surface expression of CD146, CD271 and SSEA-4. In some embodiments, the MSCs are devoid of surface expression of stage-specific embryonic antigen-4 (SSEA-4). In some embodiments, the MSCs are devoid of surface expression of stage-specific embryonic antigen-3 (SSEA-3). In some embodiments, the MSCs are devoid of surface expression of CD133. In some embodiments, the MSCs are devoid of surface expression of vascular cell adhesion protein 1 (VCAM-1 or CD106). In some embodiments, the MSCs are devoid of surface expression of CD146. In some embodiments, the MSCs are devoid of surface expression of nerve growth factor receptor (NGFR, LNGFR or CD271). In some embodiments, the MSCs are devoid of surface expression of intercellular adhesion molecule 1 (ICAM-1 or CD54). In some embodiments, the MSCs are devoid of surface expression of CD58. In some embodiments, the MSCs are devoid of surface expression of L-selectin (CD62L). In some embodiments, the MSCs are devoid of surface expression of CD9. In some embodiments, the MSCS are devoid of surface expression of at least 1, 2, 3, 4, 5, 6, 7, 8, 9 or 10 of SSEA-4, SSEA-3, CD133, CD106, CD146, CD271, CD54, CD58, CD62L and CD9.

In some embodiments, upregulation, increase or enhancement is an increase of at least 50, 75, 80, 90, 100, 110, 120, 125, 130, 140, 150, 200, 300, 400, 500, 600, 700, 800, 900, or 1000%. Each possibility represents a separate embodiment of the invention. In some embodiments, upregulation, increase or enhancement is an increase of at least 100%. In some embodiments, upregulation, increase or enhancement is an increase of at least a doubling of expression. In some embodiments, upregulation, increase or enhancement is a de novo expression.

In some embodiments, expression is mRNA expression. In some embodiments, expression is protein expression. In some embodiments, protein expression is secreted protein expression. In some embodiments, protein expression is secreted protein levels. In some embodiments, protein expression is protein secretion. In some embodiments, protein expression is surface protein expression.

Methods of detecting mRNA levels/expression, protein levels/expression, protein secretion and protein surface expression are well known in the art and any such method may be employed including the methods provided hereinbelow. Examples of such methods, include but are not limited to PCR, northern blotting, in situ hybridization, microarrays, whole genome sequencing, next generation sequencing, immunostaining, western blotting, ELISA, and proteomics arrays.

In some embodiments, the population comprises at least 1×10^7 MSCs. In some embodiments, the population comprises at least 1×10\hat{ }4, 1×10\hat{ }5, 1×10\hat{ }6, 1×10\hat{ }7, 1×10\hat{ }8, 1×10\hat{ }9, or 1×10\hat{ }10 MSCs. Each possibility represents a separate embodiment of the invention. In some embodiments, the population comprises an expanded number of MSCs as compared to a bone marrow aspirate sample. In some embodiments, expanded is by at least 2, 3, 4, 5, 6, 7, 8, 9, 10, 20, 30, 40, or 50-fold. Each possibility represents a separate embodiment of the invention.

In some embodiments, the MSC population is a pure population. In some embodiments, the MSC population is an essentially pure population. In some embodiments, the MSC population is a substantially pure population. In some embodiments, the MSC population is devoid of non-MSC cells. In some embodiments, the population comprises at least 50, 60, 70, 75, 80, 90, 95, 97, 99 or 100% MSCs. Each possibility represents a separate embodiment of the invention. In some embodiments, the population comprises at least 90% MSCs. It will be understood by a skilled artisan that any or all of these markers can be combined in any way to define the MSC population. That is the population may be defined by any combination of positive and negative markers, as well as any combination of unique markers and modulated markers.

In some embodiments, the MSC population is produced by a method of the invention. In some embodiments, the MSC population is produced by an enhancement method as is disclosed hereinbelow. In some embodiments, the MSC population is produced by a method of in vitro culture of the invention. In some embodiments, the MSC population is produced by a method of in vitro culture as is disclosed hereinbelow.

### Pharmaceutical Compositions

By another aspect, there is provided a pharmaceutical composition comprising an MSC population of the invention.

In some embodiments, the pharmaceutical composition comprises an in vitro population of MSCs. In some embodiments, the pharmaceutical composition comprises an eMSC population. In some embodiments, the pharmaceutical composition further comprises a pharmaceutically acceptable excipient, carrier or adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is formulated for administration to a subject. In some embodiments, the subject is a mammal. In some embodiments, the subject is a human. In some embodiments, the subject is a human suffering from a disease or condition treatable by MSC therapy. In some embodiments, the MSC therapy is MSC administration. In some embodiments, the pharmaceutical composition is formulated for intravenous (IV) administration. In some embodiments, the pharmaceutical composition is formulated for intrathecal (IT) administration. In some embodiments, the pharmaceutical composition is formulated for intramuscular (IM) administration.

According to another aspect, there is provided a method of treating a subject comprising administering a population of the invention or a pharmaceutical composition of the invention to the subject, thereby treating the subject.

In some embodiments, the subject is a subject in need of a method of the invention. In some embodiments, the subject is a subject in need of treatment. In some embodiments, the subject suffers from a disease or condition treatable by MSCs or MSC therapy. In some embodiments, the disease is MS. In some embodiments, the disease is ALS. In some embodiments, the subject is naive to MSC therapy. In some embodiments, the disease is a neurological disease. In some embodiments, the disease is a disease characterized by axonal death. In some embodiments, the disease is a disease characterized by elevated NfL levels. In some embodiments, the levels are in the central nervous system (CNS). In some embodiments, the levels are in the cerebral spinal fluid (CSF).

In some embodiments, treating comprises reducing NfL levels in the subject. In some embodiments, the reducing is reducing NfL levels in the blood of the subject. In some embodiments, the reducing is reducing NfL levels in the serum of the subject. In some embodiments, the reducing is reducing NfL levels in the CSF of the subject. In some embodiments, the method further comprises receiving a sample from the subject and measuring NfL levels in the sample. In some embodiments, the method further comprises receiving a sample from the subject and confirming reduced levels of NfL in the sample. In some embodiments, the sample is a bodily fluid. In some embodiments, the sample is selected from blood and serum. In some embodiments, the sample is selected from CSF, blood and serum.

In some embodiments, treating comprises improving a score on the amyotrophic lateral sclerosis functional rating scale. In some embodiments, improving a score is increasing the score. In some embodiments, improving a score is decreasing the rate of the scores decreasing. In some embodiments, treating is slowing the rate of degradation. In some embodiments, treating comprises improving at least one of speech, salivation, swallowing, handwriting, cutting, dressing/hygiene, turning in bed, walking, climbing stairs and breathing. In some embodiments, treating comprises improving speech, salivation, swallowing, handwriting, cutting, dressing/hygiene, turning in bed, walking, climbing stairs or breathing. Each possibility represents a separate embodiment of the invention. IN some embodiments, the improvement is improvement in breathing. In some embodiments, breathing is measured by a forced vital capacity (FVC) lung test. In some embodiments, treating is decreasing morbidity. In some embodiments, treating is enhancing survival.

In some embodiments, the administration is systemic administration. In some embodiments, the administration is to the CNS. In some embodiments, the administration is intrathecal administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intrathecal administration of a therapeutically effective amount of a composition of the present subject matter to a patient in need thereof. Other suitable routes of administration can include parenteral, subcutaneous, oral, intramuscular, intravenous or intraperitoneal.

The dosage administered will be dependent upon the age, health, and weight of the recipient, kind of concurrent treatment, if any, frequency of treatment, and the nature of the effect desired.

### Methods of Culture

By another aspect, there is provided a method of culturing MSCs, the method comprising,
a. receiving a cell sample comprising MSCs;
b. isolating MSCs from the sample; and
c. culturing the MSCs in media for a time sufficient for increasing MSC number;
   thereby culturing MSCs.

In some embodiments, the method is an in vitro method. In some embodiments, the method is an ex vivo method. In some embodiments, the method is a method of producing eMSCs. In some embodiments, the method is a method of producing therapeutic MSCs. In some embodiments, the method is a method of enhancing MSCs. In some embodiments, the method is a method of enhancing the therapeutic potential of MSCs. In some embodiments, the method is a method of producing an MSC population. In some embodiments, the MSC population is a therapeutic population or a population for therapy.

In some embodiments, the cell sample is a primary cell sample. In some embodiments, the cell sample is bone marrow aspirate. In some embodiments, the sample is from bone marrow. In some embodiments, the sample is from a subject. In some embodiments, the subject is a patient. In some embodiments, the subject is a healthy subject. In some embodiments, the subject is a subject in need of MSC treatment.

In some embodiments, the method comprises producing a single cell suspension from the sample. In some embodiments, method comprises homogenizing the sample. In some embodiments, isolating comprises isolating mononuclear cells (MNCs). In some embodiments, the isolating comprises isolating MSCs. In some embodiments, isolating comprises isolating adherent cells. In some embodiments, adherent cells are cells that adhere to a surface in culture. In some embodiments, the surface is a tissue culture container. In some embodiments, the container is a dish. In some embodiments, the container is a flask. In some embodiments, the method comprises placing the sample in culture. In some embodiments, the culture is a tissue culture. In some embodiments, the culture is in adherent plates. In some embodiments, isolating comprises a density gradient separation. In some embodiments, isolating comprises marker-based separation. In some embodiments, isolating comprises a positive selection. In some embodiments, isolating comprises a negative selection. In some embodiments, isolating comprises performing a Ficoll density gradient separation. In some embodiments, isolating comprises contacting the sample with a Ficoll density gradient. In some embodiments, isolating comprises Sepax separation. In some embodiments, the Sepax separation is separation of MSCs. In some embodiments, the Sepax separation is separation of MNCs. In some embodiments, isolating comprises Ficoll density gradient separation and Sepax separation. In some embodiments, the Sepax separation follows the Ficoll separation.

Sepax separation is well known in the art, and any method of Sepax separation may be employed. Examples of protocols for Sepax separation of MSCs can be found in Aktas et al., 2008 “Separation of adult bone marrow mononuclear cells using the automated closed separation system Sepax”, Cytotherapy, Vol 10 (2): 203-211; and Guven et al., 2012, “Validation of an automate procedure to isolate human adipose tissue-derived cells by using the Sepax technology”, Tissue Eng. Part C Methods, 18 (8): 575-582, herein incorporated by reference in their entirety.

In some embodiments, the method further comprises freezing the cell sample. In some embodiments, the method further comprises freezing the isolated MSCs. In some embodiments, freezing comprises placing the cell sample or isolated MSCs in freezing solution. In some embodiments, the freezing solution comprises DMSO. In some embodiments, the freezing solution comprises about 10% DMSO. In some embodiments, the freezing solution comprises at least 10% DMSO. In some embodiments, the freezing solution comprises FBS. In some embodiments, the freezing solution is about 90% FBS and 10% DMSO. In some embodiments, the freezing solution is CTS™ Synth-a-Freeze™ Medium (Thermo). In some embodiments, the freezing solution is a chemically defined media. As used herein, the term “chemically defined media” refers to a medium in which all the chemical components are known. In some embodiments, chemically defined media is devoid of animal-based products. In some embodiments, chemically defined media is devoid of animal-based proteins. In some embodiments, the freezing solution is protein free media. In some embodiments, the method further comprises thawing the cell sample. In some embodiments, the method further comprises thawing the isolated MSCs. In some embodiments, the method further comprises washing the MSCs. In some embodiments, the washing is washing the thawed MSCs. In some embodiments, the washing is with a wash solution. In some embodiments, the wash solution is PBS or DPBS. In some embodiments, the wash solution comprises PBS or DPBS. In some embodiments, the wash solution is a dextran and albumin wash solution. In some embodiments, the wash solution comprises dextran. In some embodiments, the wash solution comprises albumin. In some embodiments, the albumin is human albumin. In some embodiments, the dextran is dextran sulfate. In some embodiments, the dextran is dextran 40. In some embodiments, the wash solution comprises 2-5% dextran. In some embodiments, the wash solution comprises 0.5-10, 0.5-9, 0.5-8, 0.5-7, 0.5-6.5, 0.5-6, 0.5-5.5, 0.5-5, 0.5-4.5, 0.5-4, 0.5-3.5, 0.5-3, 1-10, 1-9, 1-8, 1-7, 1-6.5, 1-6, 1-5.5, 1-5, 1-4.5, 1-4, 1-3.5, 1-3, 1.5-10, 1.5-9, 1.5-8, 1.5-7, 1.5-6.5, 1.5-6, 1.5-5.5, 1.5-5, 1.5-4.5, 1.5-4, 1.5-3.5, 1.5-3, 2-10, 2-9, 2-8, 2-7, 2-6.5, 2-6, 2-5.5, 2-5, 2-4.5, 2-4, 2-3.5, 2-3, 2.5-10, 2.5-9, 2.5-8, 2.5-7, 2.5-6.5, 2.5-6, 2.5-5.5, 2.5-5, 2.5-4.5, 2.5-4, 2.5-3.5, 2.5-3, 3-10, 3-9, 3-8, 3-7, 3-6.5, 3-6, 3-5.5, 3-5, 3-4.5, 3-4, or 3-3.5% dextran. Each possibility represents a separate embodiment of the invention. In some embodiments, the wash solution comprises 3-10% albumin. In some embodiments, the wash solution comprises 1-15, 1-14, 1-13, 1-12, 1-11, 1-10, 1-9, 1-8, 1-7, 1-6, 1-5, 2-15, 2-14, 2-13, 2-12, 2-11, 2-10, 2-9, 2-8, 2-7, 2-6, 2-5, 3-15, 3-14, 3-13, 3-12, 3-11, 3-10, 3-9, 3-8, 3-7, 3-6, 3-5, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, or 5-6% albumin. Each possibility represents a separate embodiment of the invention.

In some embodiments, the culturing comprises a reduced seeding density as compared to a standard protocol. In some embodiments, a reduced seeding density is a density of between 5000-8000 cell/cm². In some embodiments, a reduced seeding density is a density of between 1000-12000, 1000-10000, 1000-9000, 1000-8000, 1000-7000, 1000-6000, 3000-12000, 3000-10000, 3000-9000, 3000-8000, 3000-7000, 3000-6000, 4000-12000, 4000-10000, 4000-9000, 4000-8000, 4000-7000, 4000-6000, 5000-12000, 5000-10000, 5000-9000, 5000-8000, 5000-7000, 5000-6000, 6000-12000, 6000-10000, 6000-9000, 6000-8000, 6000-7000, 7000-12000, 7000-10000, 7000-9000, or 7000-8000 cell/cm². Each possibility represents a separate embodiment of the invention.

In some embodiments, the media comprises FBS. In some embodiments, the media comprise 5-10% FBS. In some embodiments, the media comprise about 10% FBS. In some embodiments, the media is MSC media. In some embodiments, the media is tissue culture media. In some embodiments, the MSC media is NutriStem media. In some embodiments, the media is DMEM. In some embodiments, the media is supplemented with FBS. In some embodiments, the media is supplemented with serum. In some embodiments, the media is chemically defined media. In some embodiments, the media is devoid of non-human proteins. In some embodiments, the media is supplemented with human platelet lysate (HPL). In some embodiments, the media is supplemented with 5-10% HPL. In some embodiments, the media is supplemented with 5-15, 5-12, 5-11, 5-10.5, 5-10, 5-9.5, 5-9, 5-8.5, 5-8, 5-7.5, 5-7, 5-6.5, 5-6, 6-15, 6-12, 6-11, 6-10.5, 6-10, 6-9.5, 6-9, 6-8.5, 6-8, 6-7.5, 6-7, 6-6.5, 7-15, 7-12, 7-11, 7-10.5, 7-10, 7-9.5, 7-9, 7-8.5, 7-8, 7-7.5, 7.5-15, 7.5-12, 7.5-11, 7.5-10.5, 7.5-10, 7.5-9.5, 7.5-9, 7.5-8.5, or 7.5-8% HPL. Each possibility represents a separate embodiment of the invention. In some embodiments, the media is supplemented with 7.5-10% HPL. In some embodiments, the media is supplemented with 7.5-15% HPL. In some embodiments, the media is supplemented with about 10% HPL.

In some embodiments, the media is supplemented with glutamine. In some embodiments, the glutamine is L-Glutamine. In some embodiments, the glutamine is Glutamax. In some embodiments, the glutamine is about 1% glutamine. In some embodiments, the media is supplemented with non-essential amino acids. In some embodiments, the non-essential amino acids is about 0.5% non-essential amino acids. In some embodiments, the media further comprises non-essential vitamins. In some embodiments, the media is supplemented with 0.1-5, 0.1-4, 0.1-3, 0.1-2, 0.1-1.5, 0.1-1, 0.1-0.5, 0.5-5, 0.5-4, 0.5-3, 0.5-2, 0.5-1.5, or 0.5-1% non-essential vitamins. Each possibility represents a separate embodiment of the invention. In some embodiments, the media is supplemented with about 0.5% non-essential vitamins. In some embodiments, the non-essential vitamins are selected from Table 1. In some embodiments, the non-essential vitamins comprise a plurality of vitamins from Table 1. In some embodiments, the non-essential vitamins comprise at least 10 vitamins from Table 1. In some embodiments, the non-essential vitamins comprise all the vitamins from Table 1.

In some embodiments, the MSCs are culture for a time sufficient for increasing MSC number by at least 50, 100, 150, 200, 250, 300, 350, 400, 450, 500, 550, 600, 700, 750, 800, 900, or 1000%. Each possibility represents a separate embodiment of the invention. In some embodiments, the MSCs are culture for a time sufficient for increasing MSC number by at least 100%. In some embodiments, the time is a time sufficient for doubling the population of MSCs. In some embodiments, the time is a time sufficient for measurable expression of an enhanced protein. In some embodiments, the time is a time sufficient for enhanced expression of an enhanced protein. In some embodiments, the time is at least 3 days. In some embodiments, the time is at least 4 days. In some embodiments, the time is at least 5 days. In some embodiments, the time is at least 6 days. In some embodiments, the time is at least 1 week. In some embodiments, the time is at least 2 weeks. In some embodiments, the time is at least 3 weeks.

In some embodiments, the culturing comprises removing 40-70% of the media. In some embodiments, the culturing comprises removing 40-80% of the media. In some embodiments, the culturing comprises removing 40-90% of the media. In some embodiments, the culturing comprises removing about 50% of the media. In some embodiments, the culturing further comprises replacing the removed media with an equal volume of fresh media. In some embodiments, the culturing does not comprises removing 100% of the media. In some embodiments, the culturing does not comprise washing. It will be understood by a skilled artisan that for the purposes of splitting or harvesting the MSCs, all the media can be removed and the cells washed (e.g. in PBS/DPBS), however, during culturing/expansion the entire media would not be removed. In some embodiments, the culturing does not comprise exposing the MSCs directly to air after the initial plating. In some embodiments, the culturing does not comprise removing 100% of the media, washing, exposing the MSCs directly to air or a combination thereof from the initial plating of the MSCs to their splitting and/or harvesting.

In some embodiments, the removing is about every 24 hours. In some embodiments, the removing is about every 48 hours. In some embodiments, the removing is about every 72 hours. In some embodiments, the removing is between 24-48 hours. In some embodiments, the removing is between 24-72 hours.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Methods

Bone marrow aspiration: Fresh bone marrow is aspirated according to the routine Medical Center procedure from the patient's iliac crest under local anesthesia and sedation by an anesthetist. Bone marrow (100 ml) is aspirated using aspiration needles into heparin containing sterile bags. The aspirated patients' bone marrow, which is the source of the MSC, is transferred immediately to the cell processing facility of the Bone Marrow Transplantation Unit. The bone marrow aspiration procedure is preceded by documentation reporting negative test results for HBV, HCV and HIV. The patient's aspirated bone marrow sample is labeled by the physician or by the attending technical assistant.

### Example 1: Standard MSC Production from Bone Marrow Aspirates

Mesenchymal stem cells (MSCs) isolated from bone marrow aspirates are under investigation as a therapeutic agent for treating a wide variety of diseases and conditions. The standard protocol calls for obtaining aspirates from human subject, often healthy donors, or the patient to be treated themselves. The aspirates are generally frozen before they are processed. MSCs are isolated by their ability to adhere to standard tissue culture plates during culture, usually over the course of at least 1 week. The MSCs are then cultured to increase their number and can then be administered to the subject to be treated. The full protocol is as follows.

Frozen cryopreserved bags containing 80-100 ml of aspirate mixed with 10% DMSO (standard for freezing aspirate) are thawed by exposure to ambient air for 7-10 minutes to allow the bag to regain elasticity. The bag is then transferred to a 37-degree Celsius water bath for rapid thawing. Immediately upon thawing the aspirate is transferred to a conical tube containing thawing solution in equal volume to the volume of the aspirate. The standard thawing solution is 3% dextran (10 grams Dextran 40 in 0.9% sodium chloride solution) and 6% human albumin in Dulbecco's phosphate buffered saline (DPBS, without Ca and Mg). The resultant solution is mixed for 2-3 minutes by continuous pipetting.

The mixed solution is centrifuged for 10 minutes, at 400g at room temperature, after which, the supernatant is removed, and the cell pellet resuspended in 100 ml of sterile, filtered DPBS. The pellet is resuspended by light pipetting and then centrifuged again for 10 minutes at 400g and room temperature. The supernatant is removed, and the cells are counted (by hemocytometer or cell counter apparatus). The pellet is then suspended in 10 ml DPBS and applied to a Ficoll density gradient (1.073 gr/ml). After centrifugation, the intermediate layer containing the mononuclear cells (MNCs) is pipetted out, transferred to a canonical tube and diluted with 30 ml DPBS. The cells were pelleted by the same centrifugation and washed two more times in DPBS.

Following washing the MNCs were resuspended in complete culture media (DMEM LG, 10% FBS, 1% L-Glutamine, 0.5% non-essential amino acids), counted and seeded into NUNC Flasks (Thermo Fischer) at a density of 50,000 MNC/cm². Culture was performed at standard tissue culture conditions (5% CO2 and 37 degrees Celsius). After a 48-hour incubation, the media was removed, the adhered cells were washed with DPBS to remove unattached cells and new complete culture media was added. Two days later this washing was repeated, and again the old media was replaced completely with new media. From then on media was exchanged every 1-2 weeks as needed. Further, cells were split when they reached ˜90% confluency. This level of confluency could be identified by the formation of colony forming units, with spindle shaped cells and a diameter of 70-180 um/cell/colony. To split cells are washed three times with 100 ml DPBS, trypsinized, spun down (7 minutes, 1000 rmp, at 4 degrees), counted and reseeded at a density of 20,000 cells/cm². Cells can be grown until a desired number is reached, and then either administered to a patient or frozen.

MSCs are useful therapeutics because they are MEW negative and do not induce an immune response. Further, MSCs have several positive anti-inflammatory and regulatory properties. Though the standard protocol produces MSCs which are therapeutically effective, production of superior MSCs can improve a wide variety of therapies. Provided herein is an improved method of MSC production from bone marrow aspirate. The produced cellular population is in fact unique as compared to the population produced by standard methods and these superior cells are herein referred to as enhanced MSCs (eMSCs).

### Example 2: eMSC Production from Bone Marrow Aspirates

The improved protocol is as follows. Bone marrow aspirates were isolated and sent for Sepax density separation of MNCs. The Sepax isolation protocols for aspirates are well known, and can be found, for example, in Aktas et al., 2008 “Separation of adult bone marrow mononuclear cells using the automated closed separation system Sepax.” herein incorporated by reference in its entirety. The Sepax protocol greatly reduces the volume of liquid containing the MNCs and also decreases the number of contaminating non-adherent cells. The MNCs were washed with DPBS and seeded at a density of 5000-8000 cells/cm², a much lower density that was used in the old protocol following Ficoll isolation. Instead of complete culture media, the cells were cultured in NutriStem MSC XF media (Biological Industries) supplemented with 10% human platelet lysate (HPL), 1% Glutamax, 0.5% non-essential amino acids, and 1% non-essential vitamins (see Table 1).

| | Concentration in the vitamin |
|---|---|
| Vitamins | buffer (±5%) (mg/L) |
| Biotin | 0.0035 |
| D-Calcium pantothenate | 2.24 |
| Choline chloride | 8.98 |
| Folic acid | 2.65 |
| i-Inositol | 12.60 |
| Niacinamide | 2.02 |
| Pyridoxal hydrochloride | 2.00 |
| Pyridoxine hydrochloride | 0.031 |
| Riboflavin | 0.219 |
| Thiamine hydrochloride | 2.17 |
| Vitamin B12 | 0.68 |

After two days, half of the culture media was removed and replaced with fresh media. The entire media was never removed, the cells were never exposed to air and the dish was never washed. Half the media was removed and replaced every 48 hours until the cells reached 90% confluence as before. The trypsinizing protocol for splitting and harvesting the cells was performed as before. The produced cells were found to be greater than 90% MSCs based on FACS analysis (less than 5% CD34, CD45 HLA-DR, or CD79 positive and greater than 90% CD73, CD90, and CD105 positive).

### Example 3: Secretome Comparison Between Standard MSCs and eMSCs

In order to characterize the eMSCs and investigate their superiority to standard MSCs, analysis of a panel of secreted proteins was carried out. Standard MSCs were generated as above in Example 1. eMSCs were generated as above in Example 2. The standard MSCs and eMSCs protocols both started with bone marrow aspirate from the same subject. MSCs were also purchased from Lonza (#PT-2501, Batch #18TL282222). The Lonza MSCs were cultured in complete culture media. After the culture protocols were completed the cells were trypsinized, and 50,000 cells of each of the three types were seeding in 200 ul of serum/additive-free media (without serum or HPL). The cells remained in culture for 48 hours and then the media was harvested for analysis on a 40 secreted-protein ELISA panel (RayBiostech).

Of the 40 secreted factors measured, 21 were completely undetectable from either the Lonza MSCs or the MSCs produced by the old protocol but were detectable in the eMSC media. These 21 proteins and their expression in the supernatant (pg/ml, after background removal) are summarized in Table 2. 12 factors were secreted by the eMSCs and at least one of the other MSCs tested but were more highly expressed in the eMSCs. These 12 enhanced proteins and their expression (pg/ml) are summarized in Table 3. Finally, 7 factors were not upregulated in the eMSCs. These 7 proteins and their expression (pg/ml) are summarized in Table 4.

TABLE 2
Proteins only secreted by the eMSCs.

| | | MSC | |
|---|---|---|---|
| | eMSC | (old) | Lonza |
| | Avg | Avg | Avg |
| BDNF | 38 | 0 | 0 |
| bFGF | 9 | 0 | 0 |
| BMP-7 | 392 | 0 | 0 |
| b-NGF | 8 | 0 | 0 |
| EG-VEGF | 57 | 0 | 0 |
| FGF-4 | 3,187 | 0 | 0 |
| FGF-7 | 181 | 0 | 0 |
| GH | 115 | 0 | 0 |
| HB-EGF | 34 | 0 | 0 |
| IGFBP-1 | 231 | 0 | 0 |
| Insulin | 6,285 | 0 | 0 |
| NGF R | 46 | 0 | 0 |
| NT-3 | 212 | 0 | 0 |
| NT-4 | 557 | 0 | 0 |
| PDGF-AA | 276 | 0 | 0 |
| PIGF | 11 | 0 | 0 |
| SCF | 59 | 0 | 0 |
| TGFa | 58 | 0 | 0 |
| TGFb3 | 68 | 0 | 0 |
| VEGF R2 | 330 | 0 | 0 |
| VEGF R3 | 339 | 0 | 0 |

TABLE 3
Proteins that are upregulated in the eMSCs.

| | | MSC | |
|---|---|---|---|
| | eMSC | (old) | Lonza |
| | Avg | Avg | Avg |
| AR | 31 | 0 | 8 |
| BMP-4 | 5,544 | 142 | 580 |
| GDF-15 | 149 | 0 | 37 |
| GDNF | 9 | 0 | 6 |
| HGF | 391 | 0 | 26 |
| IGFBP-2 | 17,955 | 0 | 9,286 |
| IGFBP-3 | 11,213 | 0 | 2,290 |
| IGFBP-4 | 8,377 | 0 | 344 |
| IGFBP-6 | 2,375 | 1,042 | 0 |
| IGF-1 | 284 | 0 | 100 |
| VEGF | 86 | 2 | 69 |
| VEGF-D | 155 | 0 | 72 |

TABLE 4
Proteins that are not upregulated in the eMSCs.

| | | MSC | |
|---|---|---|---|
| | eMSC | (old) | Lonza |
| | Avg | Avg | Avg |
| BMP-5 | 692 | 0 | 1,262 |
| EGF | 0 | 0 | 0 |
| EGF R | 0 | 0 | 0 |
| MCSF R | 38 | 0 | 61 |
| OPG | 348 | 321 | 290 |
| SCF R | 0 | 0 | 0 |
| TGFb1 | 0 | 0 | 0 |

### Example 4: Effect of HPL and Vitamins

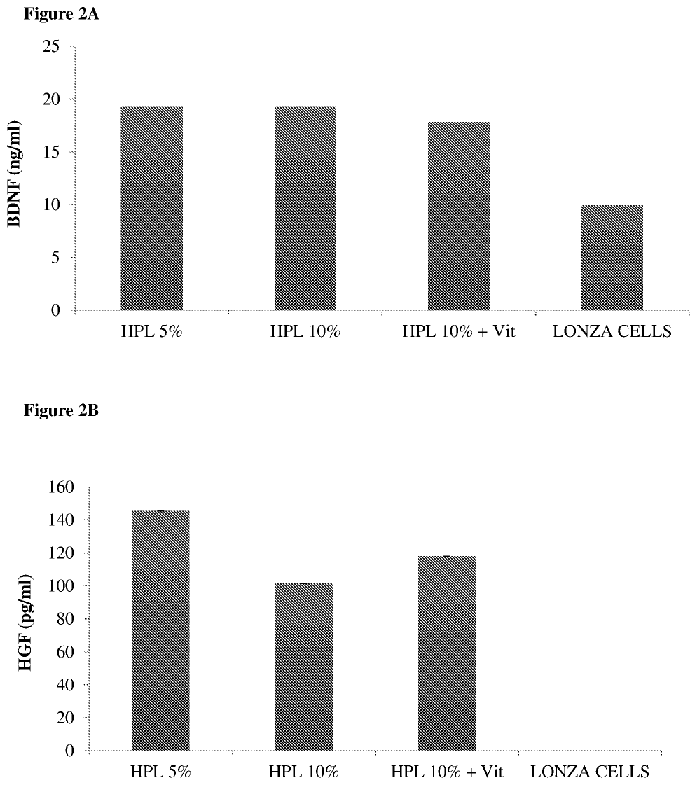

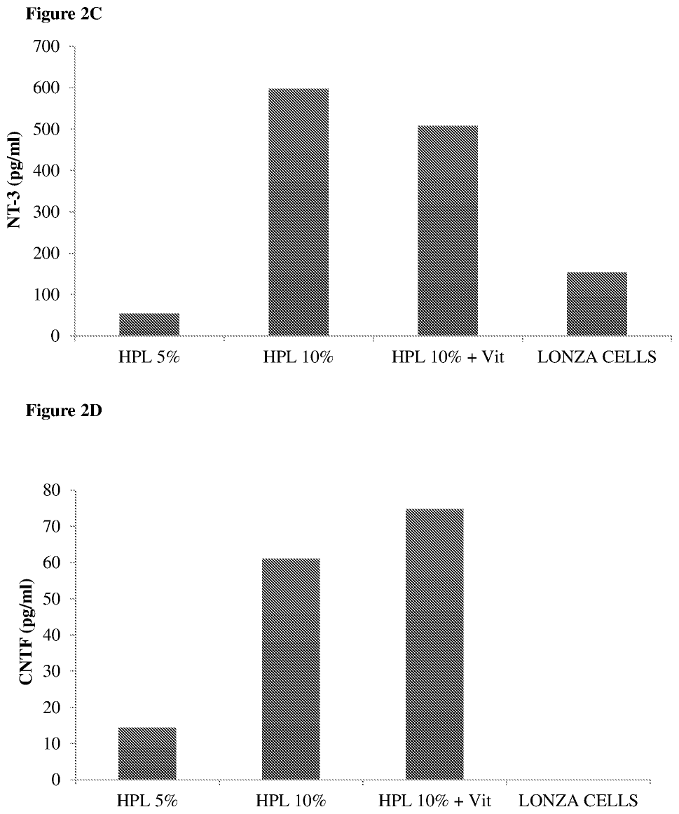

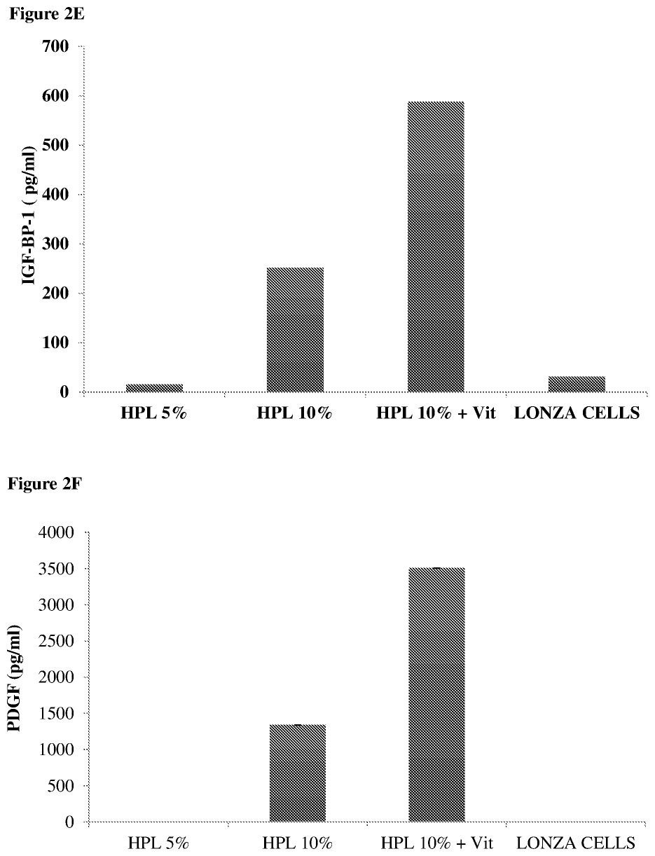

In an effort to understand how the concentration of HPL used in culture and the inclusion of the vitamins of Table 1 effect expression of secreted factors, Lonza MSCs were again compared to eMSCs. In this experiment the eMSCs were produced either with 5% HPL but without vitamins, with 10% HPL without vitamins or with 10% HPL and vitamins (the protocol from Example #2). Secretion of 6 factors was measured by ELISA and the results are summarized in FIGS. 2A-2F. The 6 factors tested were BDNF, HGF, NT-3, CNFT, IGF-BP1 and PDGF, all of which were found to either be uniquely secreted by the eMSCs or to be upregulated in eMSCs.

BDNF was found to be more lowly expressed in the Lonza cells as compared to all 3 of the tested eMSCs, however, no difference was observed between 5% HPL, 10% HPL and 10% HPL with vitamins (FIG. 2A). HGF was not expressed at all in the Lonza cells but was strongly upregulated in all tested eMSCs (FIG. 2B). Again, no difference was observed between the various produced eMSCs.

For NT-3 a different pattern was observed. eMSCs produced with 5% HLP showed comparable, or even lower levels of NT-3 as compared to the Lonza cells, however when 10% HPL was used the expression of NT-3 was increased by 3-fold (FIG. 2C). No significant difference in NT-3 levels was observed when the vitamins were included.

CNTF was not secreted by the Lonza cells but was detected from all 3 produced eMSCs (FIG. 2D). 5% HPL produced a modest secretion of CNTF, but the use of 10% HPL greatly increased CNTF expression (more than 5-fold over 5%). Inclusion of vitamins produced a small increase in CNTF expression.

IGF-BP-1 was very lowly secreted by the Lonza cells and eMSC produced with 5% HPL, however, use of 10% HPL increased the secretion of IGF-BP-1 by close to 10-fold (FIG. 2E). In this case the addition of vitamins produced a robust increase in secretion, more than doubling the secretion as compared to 10% HPL without vitamins. Similar results were observed for PDGF. PDGF was not secreted by the Lonza cells or the eMSCs produced with 5% HPL but was highly secreted by the eMSCs produced with 10% HPL and no vitamins (FIG. 2F). The addition of the vitamins, however, more than doubled secretion of PDGF. This data taken together shows that 10% HPL is unexpectedly superior to 5% HPL, not merely increasing secretion of several important factors, but actually inducing expression of new factors. Further, the addition of the vitamins was surprisingly beneficial more than doubling the secretion of several factors.

### Example 5: Surface Protein Expression in eMSCs

The eMSCs (protocol from Example #2) were analyzed by FACS analysis to determine the presence of known surface proteins. First, surface markers known to be expressed by MSCs, and in particular bone marrow MSC, were analyzed. Though the cells were greater than 90% positive for CD73, CD90, and CD105, staining for SSEA-4, SSEA-3, CD133, CD106, CD146, CD271, CD54, CD58, CD62L and CD9 found the cells to be completely negative for these markers. That is 100% of the cells were negative for all 10 of these markers. In contrast, MSCs produced by the standard protocol were found to be 67.3% positive for CD146, 60.1% positive for CD271 and 48.5% positive for SSEA-4. Next, surface expression of 27 proteins not known to be expressed by MSCs was measured. The results are summarized in Table 5.

TABLE 5
Ectopic surface proteins expressed by eMSCs.

| | % Positively |
|---|---|
| Marker | Stained Cells |
| SSEA-5 | 85.86% |
| NPC (57D2) | 83.81% |
| MUC-13 | 78.49% |
| CD206 | 74.10% |
| Notch1 | 72.66% |
| Notch4 | 70.18% |
| Notch3 | 67.33% |
| NTBA | 61.64% |
| NKp80 | 59.17% |
| CD207 | 58.83% |
| CD132 | 53.60% |
| Jagged 2 | 48.72% |
| GPR-56 | 47.85% |
| CD66 | 45.71% |
| DR3 | 43.57% |
| CD85j | 41.05% |
| CD183 | 40.70% |
| CD85h | 38.89% |
| CD319 | 36.87% |
| GPR-19 | 35.17% |
| CD24 | 33.99% |
| HVEM | 32.70% |
| EGF-R | 32.19% |
| CD309 (VEGFR2) | 32.09% |
| CD314 | 32.03% |
| BTLA | 31.95% |
| CD368 | 31.61% |

This level of surface expression was much higher that what would be expected based on publication of MSC, and in particular bone marrow MSC, surface expression. Indeed, no expression at all would have been expected. To confirm this, the surface expression of 6 of the proteins was examined in MSC cultured by the standard protocol. The results are summarized in Table 6.

TABLE 6
Surface proteins expression by standard MSCs

| | % Positive |
|---|---|
| Marker | in MSC (old) |
| CD146 | 67.30% |
| CD271 | 60.10% |
| SSEA-4 | 48.50% |
| SSEA-5 | 0.20% |
| NPC | 13.30% |
| MUC13 | 13.50% |
| NOTCH-1 | 0.00% |
| CD207 | 5.90% |
| NTBA | 0.00% |

The standard MSCs were found to be completely negative for NOTCH-1 and CD352 and nearly so for SSEA-5 (the 0.20% positivity may have been none specific expression). NPC, MUC13 and CD207 did show some positive cells, though never more than 15% of the population. In contrast, the eMSCs showed much higher levels of these proteins, with an at least 5× increase in expression for all of them.

The significance of the HPL concentration, the SEPAX isolation, the non-essential vitamins, the seeding density, and the washing schedule is tested. Each of these components in the eMSC protocol is altered to be the same as the standard protocol and at least one eMSC marker is measured. Alterations that result in the eMSC marker expression returning to the expression level found in standard MSCs is considered an essential component of the protocol.

### Example 6: Treatment of Multiple Sclerosis Patient with eMSCs

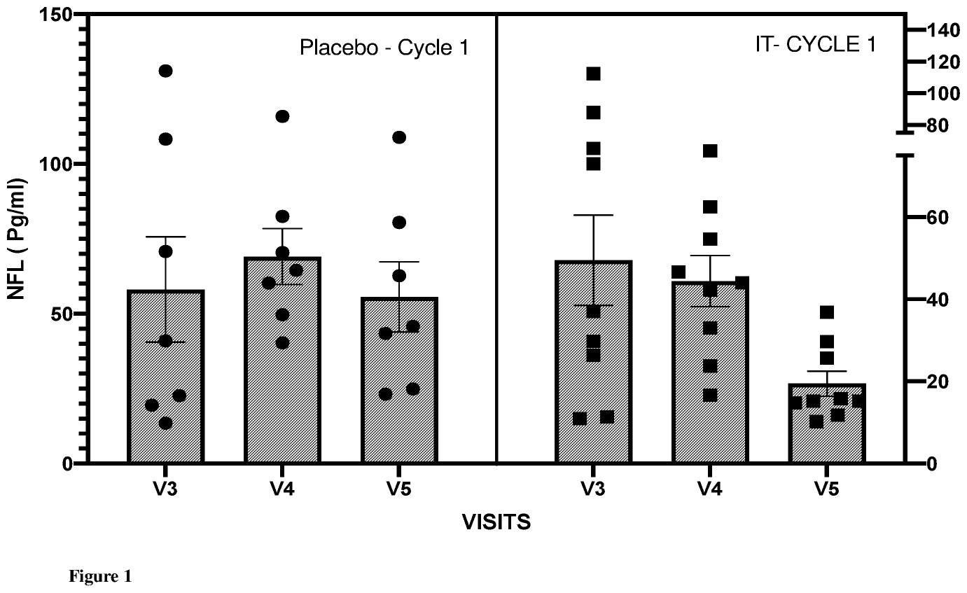

In order to test the efficacy of the eMSCs, 16 Multiple Sclerosis (MS) patients from the Hadassah MS center were selected for treatment with the eMSCs or placebo. The patients had not received any immunomodulatory treatment during at least the previous year. 9 of the patients received eMSCs (1×10\hat{ }6/Kg) via intrathecal (IT) administration, while 7 received IT administration of placebo. In order to monitor neurodegeneration, the level of neurofilament light chain (NfL) in serum was monitored using SIMOA® TECHNOLOGY. Patients were measured at 3 separate time points: 24 hours before administration (V3), 1 month after administration (V4) and 2 months after administration (V5). As can be seen in FIG. 1, at the initial time point the NfL levels were comparable in the treated and placebo groups. At the second measuring the placebo group has seen a small increase while the treatment group has seen a small decrease in levels. Most strikingly, at the final time point the levels in the placebo group remain unchanged, whereas the treated group saw a significant reduction in NfL levels (p<0.05). This reduction was superior to the reduction produced by MSCs produced the standard method described hereinabove.

### Example 7: Treatment of ALS Patient with eMSCs

In order to test the efficacy of the eMSCs, amyotrophic lateral sclerosis (ALS) patients from the Hadassah MS center were selected for compassionate treatment with the eMSCs. The patients received 1-4 injections of eMSCs (1×10\hat{ }6/Kg) via intrathecal (IT) administration. Injections were spread out every 3-6 months. After the MSC transplantation patients were examined on a bimonthly basis and evaluated with the Amyotrophic Lateral Sclerosis Functional Rating Scale (ALSFRS) and for Forced Vital Capacity (FVC) for a total post (last) treatment follow up period of 6 months. Most patients were found to have a greater than 25% improvement in ALSRFS and many improved clinically. Improvement was found to be greater than that observed using MSCs produced by the old protocol.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An in vitro population of enhanced mesenchymal stem cells (eMSCs), comprising expression of insulin like growth factor binding protein 1 (IGFBP-1), and neurotrophin 3 (NT-3).

2. (canceled)

3. The in vitro population of eMSCs of claim 1, further comprising surface expression of NPC intracellular cholesterol transporter 1 (NPC1), and CD206.

4. The in vitro population of eMSCs of claim 1, devoid of surface expression of at least one of protein selected from: CD271, SSEA-4, SSEA-3, CD133, CD106, CD146, CD54, CD58, CD62L and CD9.

5. (canceled)

6. The in vitro population of claim 1, wherein said population is characterized by enhanced pro-neurogenic capacity, enhanced immunosuppression, enhanced immunomodulation, enhanced anti-inflammatory capacity, enhanced pro-angiogenic capacity, enhanced neuroprotection, enhanced anti-apoptotic capacity, enhanced myelinogenic capacity, enhanced anti-fibrotic capacity, enhanced oligodendrocyte support, enhanced axonal support, enhanced neuronal differentiation or a combination thereof.

7. (canceled)

8. (canceled)

9. (canceled)

10. (canceled)

11. The in vitro population of claim 1, wherein at least one of:
a. said expression is protein secretion;
b. said population comprises at least 1×10\hat{ }7 MSCs;
c. said MSCs are human MSCs;
d. said MSCs are bone marrow derived MSCs; and
e. said population comprise at least 90% MSCs.

12. (canceled)

13. (canceled)

14. (canceled)

15. (canceled)

16. The in vitro population of claim 3, wherein a first subpopulation of said in vitro population comprises
at least 80% of the eMSCs and the surface protein NPC,
and a second subpopulation comprises at least 70% of the eMSCs and the surface protein CD206.

17. (canceled)

18. A method of culturing MSCs, the method comprising,
a. receiving a primary cell sample from a subject comprising MSCs;
b. isolating MSCs from said sample; and
c. culturing said MSCs in media for a time sufficient for increasing MSC number by at least 100%;
wherein at least one of the following:
i. said isolating comprises isolating mononuclear cells (MNCs) by Sepax separation;
ii. said culturing comprises an initial seeding density of between 5000-8000 cells/square centimeter;
iii. said media is NutriStem media supplemented with 5-15% human platelet lysate (HPL); or
iv. a combination thereof;
thereby culturing MSCs.

19. The method of claim 18, wherein at least one of:
a. said primary cell sample is bone marrow aspirate;
b. said isolating comprises isolating mononuclear cells (MNCs);
c. said isolating comprises isolating MNCs by performing a Ficoll density gradient, Sepax separation or both;
d. said method further comprises freezing said isolated MSCs and thawing said isolated MSCs;
e. said method further comprises freezing said isolated MSCs and thawing said isolated MSCs and washing said thawed MSCs is a Dextran and albumin wash solution, optionally wherein said wash solution comprises from 2-5% dextran 40 and 3-10% human Albumin;
f. said culturing comprises an initial seeding density of between 5000-8000 cells/square centimeter;
g. said time is at least 4 days;
h. said culturing comprises removing 40-70% of said media and replacing it with an equal volume of fresh media about every 48 hours; and
i. said method is a method of producing an in vitro population of claim 1.

20. (canceled)

21. (canceled)

22. (canceled)

23. (canceled)

24. (canceled)

25. (canceled)

26. The method of claim 18, wherein said media is NutriStem media supplemented with human platelet lysate (HPL), optionally wherein said NutriStem media is supplemented with 7.5 to 15% HPL, said media is further supplemented with non-essential vitamins, non-essential amino acids or both or said media is further supplemented with at least one non-essential vitamins selected from Table 1.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A pharmaceutical composition comprises the in vitro population of claim 1.

42. The pharmaceutical composition of claim 41, formulated for administration to a subject, intravenous administration, intrathecal administration or a combination thereof.

43. (canceled)

44. A method of treating a subject suffering from a condition treatable by MSC therapy, the method comprising administering to said subject the pharmaceutical composition of claim 41.

45. The method of claim 44, wherein said condition is multiple sclerosis (MS).

46. The method of claim 44, wherein said condition is amyotrophic lateral sclerosis (ALS).

47. The method of claim 44, wherein said treating comprises decreasing neurofilament light chain (NfL) expression in serum said subject.

48. (canceled)
